(19) United States
(12) Patent Application Publication
(10) Pub. No.: US 2014/0222020 A1
(43) Pub. Date: Aug. 7, 2014

(54) TELESCOPING REINFORCEMENTS FOR INSTRUMENT CHANNEL SHAFTS

(71) Applicant: TransEnterix, Inc., Morrisville, NC (US)

(72) Inventors: Nicholas J. Bender, Raleigh, NC (US); Matthew Robert Penny, Holly Springs, NC (US); Bryan J. Peters, Cary, NC (US); Nicholas J. Jardine, Cary, NC (US)

(73) Assignee: TransEnterix, Inc., Morrisville, NC (US)

(21) Appl. No.: 14/084,615

(22) Filed: Nov. 20, 2013

Related U.S. Application Data
(60) Provisional application No. 61/728,296, filed on Nov. 20, 2012.
(60) Provisional application No. 61/801,781, filed on Mar. 15, 2013.

(51) Int. Cl. A61B 17/00

(52) U.S. Cl. CPC A61B 17/00234; A61B 2017/00336
USPC 606/130

(57) ABSTRACT

An instrument channel device for receiving a medical instrument having a distal tip includes an elongate tubular shaft including a proximal shaft portion and a distal shaft portion positionable within a body cavity. The shaft defines a lumen for passage of the distal tip of a flexible medical instrument therethrough, allowing positioning of the medical instrument such that its distal tip is disposed distally of the distal shaft portion. A telescoping reinforcement is positioned on the distal shaft portion. The reinforcement has a collapsed position in which the telescoping reinforcement has a length that is shorter than the length of the telescoping reinforcement in an expanded position. As an instrument is advanced from the distal end of the lumen, the telescoping reinforcement is engaged by a distal portion of the instrument such that advancement of the distal tip to a position beyond a distal end of the telescoping reinforcement moves the telescoping reinforcement to the expanded position. The reinforcement minimizes unintended flexion of the portion of the instrument shaft that extends beyond the distal end of the lumen.

This application claims the benefit of U.S. Provisional Application No. 61/728,296, filed Nov. 20, 2012, and U.S. Provisional Application No. 61/801,781, filed Mar. 15, 2013, each of which is incorporated herein by reference.

### TECHNICAL FIELD OF THE INVENTION

The present invention relates generally to the field of steerable channel devices for minimally invasive surgical procedures, and more particularly to reinforcements for minimizing unintended deflection of the shafts flexible instruments steered by steerable channel devices during use.

### BACKGROUND

Surgery in the abdominal cavity is frequently performed using open laparoscopic procedures, in which multiple small incisions or ports are formed through the skin and underlying muscle and peritoneal tissue to gain access to the peritoneal site using the various instruments and scopes needed to complete the procedure. The peritoneal cavity is typically inflated using insufflation gas to expand the cavity, thus improving visualization and working space. Further developments have led to systems allowing such procedures to be performed using fewer ports, and in some cases only a single port.

Some instrument access devices or ports suitable for use in single port procedures and other laparoscopic procedures are described in co-pending U.S. application Ser. No. 11/804,063 (US Publication 2007-0299387) filed May 17, 2007 and entitled SYSTEM AND METHOD FOR MULTI-INSTRUMENT SURGICAL ACCESS USING A SINGLE ACCESS PORT, U.S. application Ser. No. 12/209,408 (US Publication 2009-227843) filed Sep. 12, 2008 and entitled MULTI-INSTRUMENT ACCESS DEVICES AND SYSTEMS, and U.S. application Ser. No. 12/511,043 (US Publication 2011-0060183) filed Jul. 28, 2009, entitled MULTI-INSTRUMENT ACCESS DEVICES AND SYSTEMS, and U.S. application Ser. No. 12/846,788 (US Publication US 2011-0184231), entitled DEFLECTABLE INSTRUMENT PORTS, filed Jul. 29, 2010, and U.S. application Ser. No. 13/651,278 (Attorney Docket: TRX-2620), entitled Deflectable Instrument Shafts, filed Oct. 12, 2012, each of which is incorporated herein by reference. The aforementioned patent applications describe access systems incorporating at least one and in some cases multiple steerable instrument channel devices.

Referring to FIG. 1 in which the distal portion of a steerable instrument channel device is shown, each such steerable instrument channel device 210 includes a lumen 220 for receiving a flexible medical instrument 230. The instrument channel device 210 includes a proximal shaft portion 202 and a steerable/deflectable portion 200. The steerable/deflectable portion 200 is actively steered using an actuator positioned proximally on the device 210 for manipulation by a user. Manipulation of the actuator engages pull elements or other actuation components coupled to the portion 200 to effect steering. Thus the instrument channel device 210 is used to support and steer the flexible medical instrument passed through it. Medical instruments that may be used through such steerable channels include, but are not limited to, flexible-shaft forceps, graspers, dissectors, electrosurgical instruments, retractors, scopes, and tissue securing devices such as suture devices or staplers.

The medical instrument is one having a flexible shaft 240 and an end effector 250 on the distal end of the flexible shaft. The medical instrument may further include a handle (not shown) on the proximal end of the shaft. The handle may include an actuator used for actuating the end effector, such as by opening/closing jaws, applying a staple or other fastener etc, or features for energizing an electrosurgical element.

In use, the distal portion of the flexible instrument is passed through the steerable instrument channel such that the end effector 250 exits the distal end of the steerable instrument channel. Deflection or steering of the flexible instrument is carried out by actively steering or deflecting the distal portion of the steerable instrument channel. The flexible instrument is removably received within the lumen of the steerable instrument channel so that it may be interchanged with other medical instruments during the course of the procedure.

In some instances, a portion of the medical instrument's flexible shaft 240 may be positioned distal to the distal end of the steerable instrument channel, and thus may undesirably flex within the body. For example, the flexible instrument shaft may flex as the end effector is engaged with tissue, contacts or advances against tissue, or is used for tissue manipulation. Such flexion may occur during times when the instrument channel is actively steered and also when the user is not actively steering the channel. The present application describes a feature that may be used at the distal end of a steerable instrument channel to minimize such unintended flexion of the flexible instrument shaft.

### BRIEF DESCRIPTION OF THE DRAWINGS

FIG. 1 is a perspective view showing the distal end portion of the shaft of a prior art steerable instrument channel device, with a flexible instrument and its end effector extending from the distal end of the shaft;

FIG. 2A is a perspective view showing the distal end portion of a first embodiment of a shaft comprising a steerable portion and a telescoping reinforcement, in which the telescoping reinforcement is shown in the collapsed position;

FIG. 2B is similar to FIG. 2A, shows the telescoping reinforcement in the extended position, and further shows an end effector of a flexible instrument extending from the distal end of the shaft;

FIG. 3 is an exploded perspective view of the telescoping reinforcement of FIGS. 2A and 2B;

FIGS. 4A and 4B are side elevation views of telescoping members of the telescoping reinforcement of FIGS. 2A and 2B;

FIG. 5 is a perspective view of the steerable portion of the shaft of FIGS. 2A and 2B;

FIGS. 6 is a perspective exploded view of three vertebrae of the steerable portion of FIG. 5;

FIG. 7 is a perspective top view of the ball vertebrae of FIG. 6;

FIG. 8 is similar to FIG. 6, but shows the vertebrae in a straight configuration;

FIG. 9A is a perspective view showing the distal end portion of a second embodiment of a shaft comprising a steerable portion and a telescoping reinforcement, in which the telescoping reinforcement is shown in the collapsed position;

FIG. 9B is similar to FIG. 9A and shows the telescoping reinforcement in the extended position.

### DETAILED DESCRIPTION

This application describes reinforcing elements positioned at a distal end of an instrument channel device through which the distal ends of medical instruments are delivered to an operative site within a body cavity. The reinforcing elements are disclosed as positioned at the end of an instrument channel device having a steerable distal portion, although they might instead be used on other types of instrument channel devices.

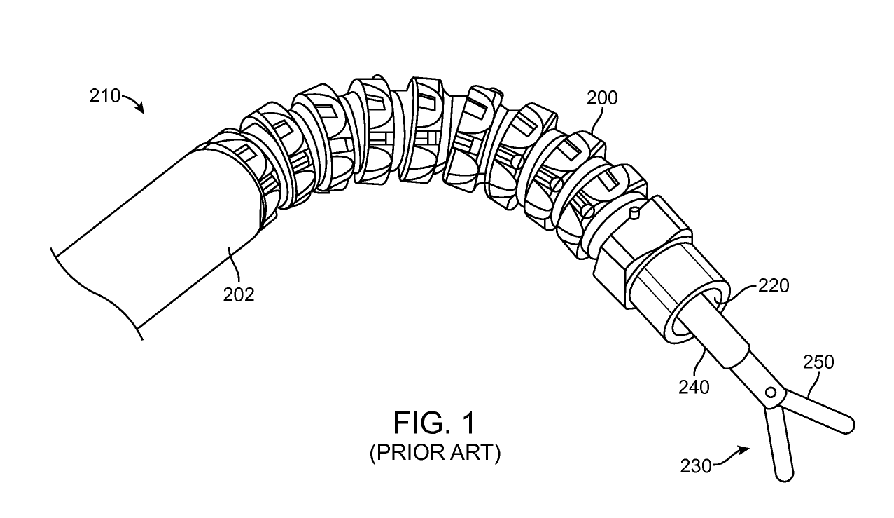

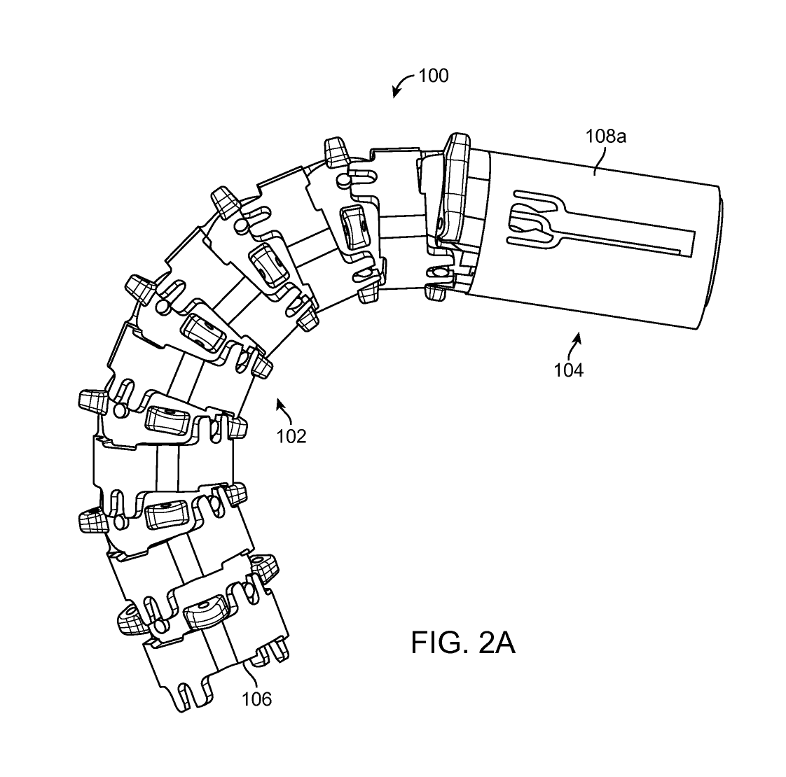

FIG. 2A is a perspective view showing a first embodiment including a shaft 100 comprising a steerable portion 102 coupled to the distal portion of a proximal shaft portion (not shown but see proximal shaft portion 202 of FIG. 1), and a telescoping reinforcement 104 disposed at the distal end of the steerable portion 102. The steerable portion 102, proximal shaft, and telescoping reinforcement define a lumen 106 for removably receiving a medical instrument. A port at the proximal end of the shaft 100 provides access for instruments into the lumen 106.

The proximal shaft portion may be a flexible, rigid, semi-rigid, or rigidizable tubular shaft. Examples are found in the co-pending patent applications referenced in the Background section above.

Telescoping reinforcement 104 is formed of a plurality of telescoping members 108a-108e having a collapsed position as shown in FIG. 2A (in which only the outermost, proximalmost, telescoping member 108a is visible) and an extended position in which the reinforcement 104 is longitudinally expanded as shown in FIG. 2B. The members 108a-108e of the first embodiment are a plurality of longitudinally-arranged tubular members, with each member shaped to nest within the proximally-adjacent member. For example, the members may be cylindrical members, each of which has an outer diameter that is smaller than the inner diameter of its proximally-adjacent neighbor. The members are formed of a rigid material such as stainless steel. Where instruments having electrosurgical tips are to be used through the instrument channel, at least the distal member 108e may be formed of a thermally insulative material such as PEEK so as to prevent the telescoping reinforcement 104 from heating during use of the electrosurgical tips.

Members 108a-108d include longitudinally extending slots or cutouts 110 in their side walls. Each member may include more than one such slots. For example, in the illustrated embodiment each member includes a pair of longitudinally extending slots 110 spaced 180° from one another. In the drawings the slots 110 in members 108a and 108c are shown off-set 90° from the slots 110 in members 108b and 108d. Different (or no) offsets may be used in other embodiments. Each slot has a distal end as shown.

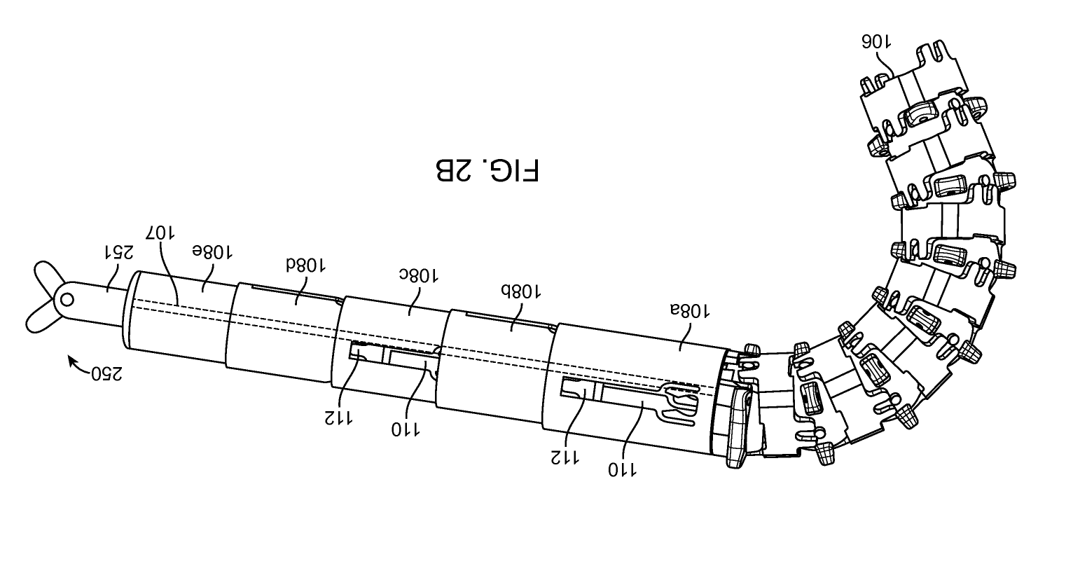

Detents 112 (best seen in FIG. 3) are formed in the side walls of members 108b-108e. When the telescoping reinforcement is in the expanded position as shown in FIG. 2B (and optionally also in the collapsed position of FIG. 2A), each detent 112 of a member 108b-108e is radially and circumferentially aligned with the slot 110 of that member's proximally adjacent neighbor 108a-108d. Detents 112 are biased in a slight radially-outward direction, such that when the telescoping reinforcement is expanded each detent 112 abuts the distal end of its corresponding slot 110, which forms a stop against further movement of the member having the detent relative to the member having the corresponding slot 110.

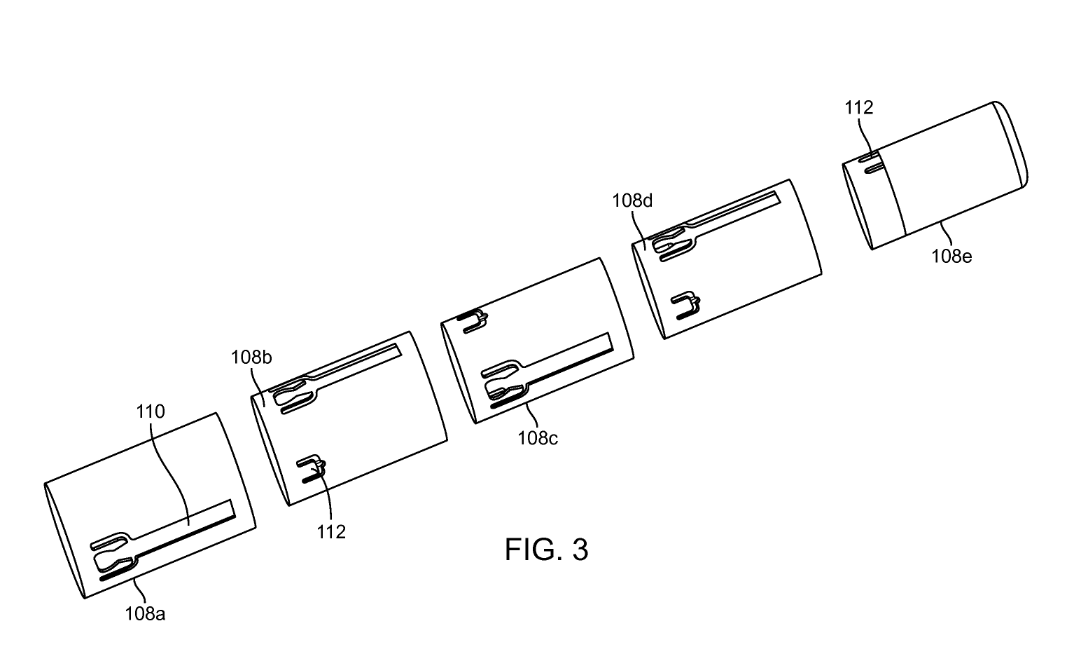

Prior to insertion of a medical instrument through the lumen 106, the members 108a-108e are arranged in the nested configuration shown in FIG. 2A. Some embodiments are equipped with features that retain the members 108a-108e in this nested configuration so that the telescoping reinforcement will only move to the expanded position of FIG. 2B when actively made to do so. This avoids inadvertent expansion of the telescoping reinforcement that might occur during movement of the steerable instrument channel (e.g. during preparation of the device for use or during insertion into the body cavity). In the FIG. 2A-4B embodiments, each of the members 108a-108d includes a pair of deflecting members 114 formed at the proximal end of each slot 110. When a telescoping member (e.g. member 108b) is moved proximally such that its detent 112 contacts deflecting members 114 of its proximally adjacent neighbor 108a, deflecting members 114 flex slightly away from one another, allowing the detent 112 to pass between them and thus capturing the detent 112 between their opposed surfaces. Each pair of deflecting members 114 is thus positioned to engage the corresponding detent 112 of the distally adjacent neighbor 108b-108e.

In alternate embodiments, magnetic features at the proximal end of the telescoping reinforcement are used to retain the members 108a-108e in the proximal, nested, configuration by engaging with magnetic material disposed on the distal member 108e.

In other alternate embodiments, elastomeric materials including, but not limited to, silicone or extension springs 107 may be terminally attached to distal member 108e such that extension of the telescoping members is opposed by a spring force exerted by the elastomeric member.

Other alternate embodiments may include a retention mechanism that may be released by rotation or other actuation means perpetrated by the distal end of the instrument shaft or end effector. In these embodiments, extension of members 108a-108e is impeded by a mechanical stop that must be engaged by the instrument end effector to be released.

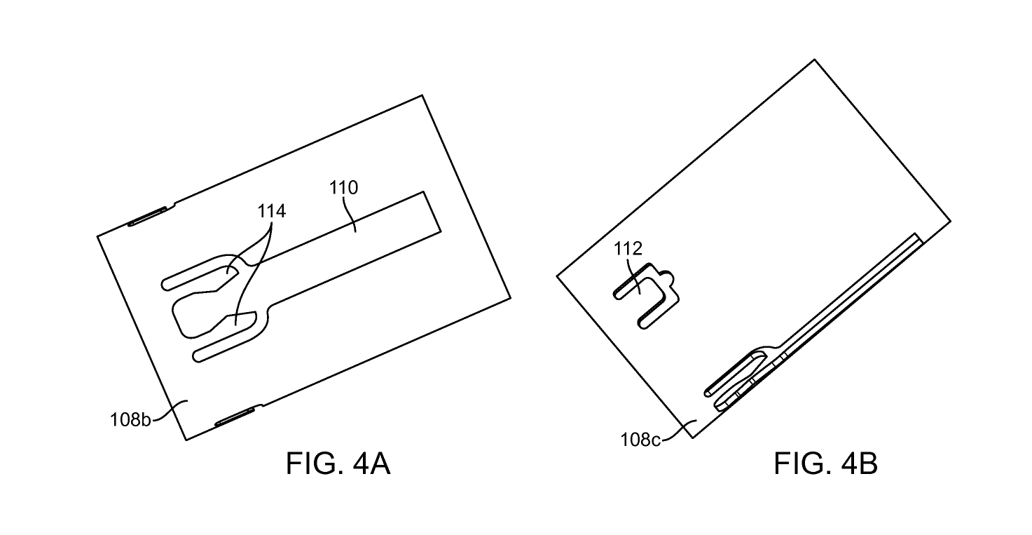

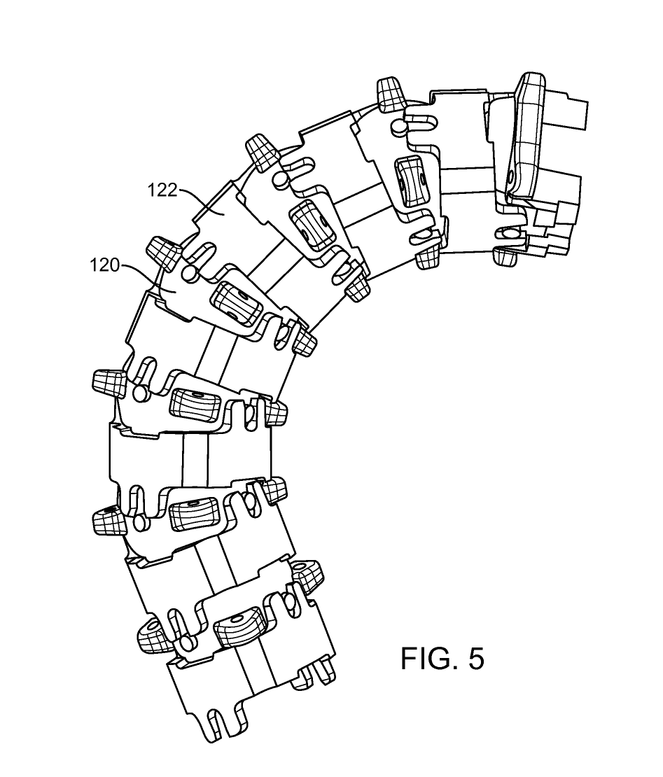

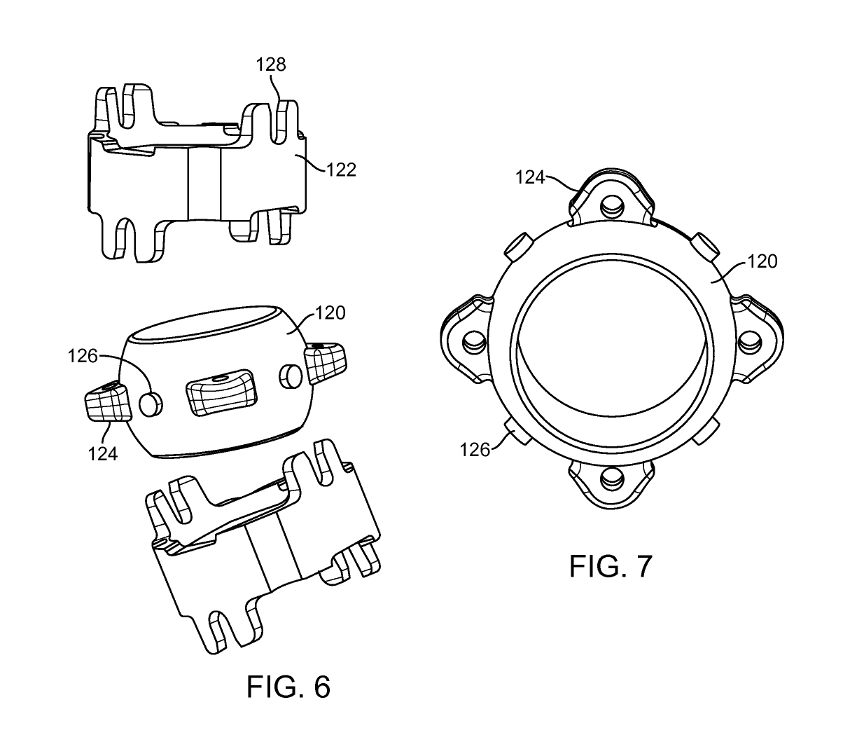

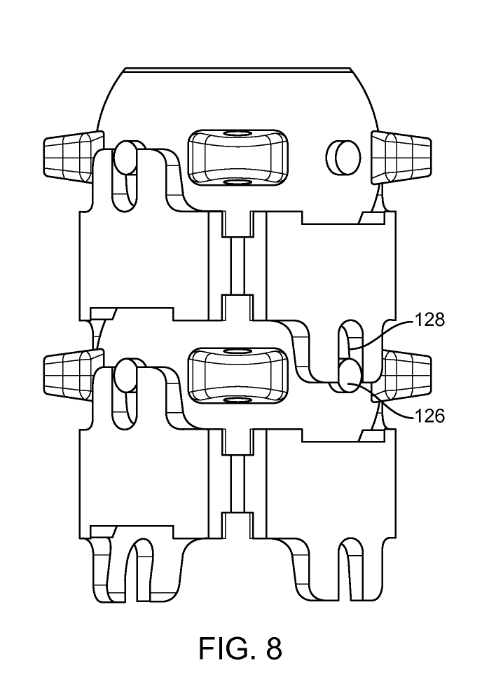

The steerable portion 102 of the instrument shaft 100 may take one of many forms suitable for use in constructing a steerable instrument shaft, including flexible tubing, vertebrae segments, slotted tubing (e.g. laser cut tubing) etc. FIGS. 5 through 8 show one type of vertebrae arrangement may be used to form the steerable portion. This arrangement uses a strand of ball segments 120 alternating with socket segments 122. Each ball segment 120 has at least a partially spherical exterior surface, and each socket segment 122 has a beveled or partially spherical interior surface for receiving a portion of the ball segment's outer surface. This arrangement forms a generally smooth and continuous lumen through the steerable portion 102, minimizing voids against which the end effector 250 (FIG. 1) of the medical instrument can catch as it is distally advanced through the lumen into the body cavity.

Pull elements (not shown) employed to steer the steerable portion 102 extend through guides 124 disposed on the exterior surface of the ball segment 120. As described in the prior applications incorporated herein, the distal ends of the pull elements are coupled to the distal end of the steerable instrument channel, and the proximal ends are coupled to an actuator that applies/releases tension on the pull elements to steer the portion 102. Examples of actuators that may be used to engage the pull elements are disclosed in the incorporated prior applications.

The number of guides 124 used preferably corresponds to the number of pull elements that are to be used to steer the steerable portion 102. Four guides 124 are shown spaced at 90 degree intervals, corresponding to the use of two or four pull elements.

The vertebrae segments include anti-rotation features to prevent the segments 120, 122 from axially rotating relative to one another. As one example, anti-rotation members on one segment are engaged by anti-rotation features on the adjacent segments. In the drawings, the ball segments 120 have anti-rotation posts 126 that are received in corresponding receivers 128 extending proximally and distally from the socket segments 122.

Other deflectable shaft features that used in combination with the telescoping tip described herein are described in U.S. application Ser. No. 13/651,278, entitled Deflectable Instrument Shafts, filed Oct. 12, 2012, which is incorporated herein by reference. For example, a tubular liner of PTFE or other material may extend longitudinally through the lumen to form a smooth passageway for movement of instruments through the shaft. A skin formed of a thin flexible membrane or material may cover the segments to prevent surrounding body tissue or other material from passing into the spaces between adjacent segments, or from being pinched or captured between adjacent segments. The skin is preferably loose enough that it will not resist deflection of the shaft when the pull elements are actuated.

During use of the first embodiment, the tip of the medical instrument (e.g. the end effector 250) is inserted into the proximal end of the lumen of instrument channel device 100, advanced through the steerable portion 102 and into the telescoping reinforcement 104 which is in the nested configuration shown in FIG. 2A. As the instrument tip exits the lumen at the distal end of the still-nested telescoping reinforcement, an engaging element on the instrument tip (e.g. on a portion of the end effector's clevis 251 or some other structure) contacts a stop on the innermost telescoping member 108e. The engaging element may also exist on the instrument shaft, more proximal to the instrument end effector to achieve additional instrument extension length. Continued advancement of the instrument tip distally pushes the member 108e sufficiently to overcome the engagement between the detent 112 of the member 108e and the deflecting members 114 of member 108d, and causes the telescoping member 108e to advance in a distal direction relative to the other members 108a-108d. Once the detent 112 of member 108e reaches the distal end of the slot 110 of member 108d, member 108e can no longer move relative to member 108d. Thus continued advancement of the instrument tip overcomes the retention forces of member 108c's deflecting members 114 on member 108d's detents 112 and extends the member 108d distally relative to the members 108a-108c. Sequential extension of the members 108b-e of telescoping reinforcement 104 continues in this manner until the most proximal extendable member 108b is fully extended. FIG. 2B shows the end effector 251 on the instrument tip positioned distally of the telescoping reinforcement for use in a body cavity, with the telescoping reinforcement 104 fully extended such that it can prevent undesirable flexing of the portion of the instrument shaft that is distal to the steerable portion 102 of the steerable channel 100.

Features may be included for retracting the members 108b-108e in a proximal direction upon withdrawal of the instrument tip or end effector 250 from the telescoping reinforcement 104. In one embodiment, a feature on the end effector 250 or clevis engages with a corresponding feature within member 108e, such that withdrawing the end effector in a proximal direction withdraws member 108e and then sequentially causes withdrawal of each of the more proximal members 108d-108b. In one embodiment, the engaging features are magnetic elements. For example, the stop within the distal end of member 108e may be a magnetic ring, and a component (e.g. a collar) on the instrument's clevis 251 is formed of a material that will magnetically engage with the magnetic ring.

An alternative arrangement for withdrawing the telescoping reinforcement 104 utilizes a flat wire coil (or a circular cross-section extension spring) wound in a tubular configuration and having a first end coupled to the member 108e and a second end coupled more proximally, such as to member 108a. The coil may be positioned in the lumen of the telescoping reinforcement, or around the telescoping reinforcement's outer surface. In this embodiment, the coil is stretched when the telescoping reinforcement 104 is in the expanded position. When the end effector is withdrawn from the telescoping reinforcement 104, the coil retracts and draws the members 108b-108e proximally into the collapsed position. It should be pointed out that the coil may apply sufficient retention forces to the members 108b-108e to prevent undesired expansion of the telescoping reinforcement 104, thus eliminating the need for the deflecting members 114 described above.

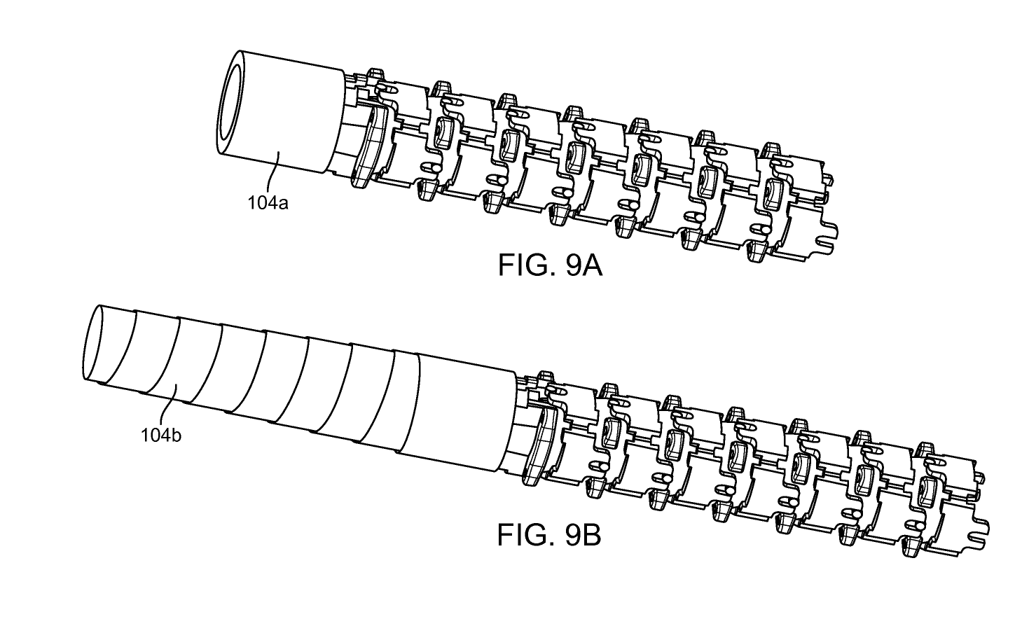

A flat wire coil of the type described in the prior paragraph may itself serve as a telescoping reinforcement, thus providing a telescoping reinforcement that will self-retract following withdrawal of the end effector, and that will remain biased in the nested configuration. As shown in FIGS. 9A and 9B, this telescoping reinforcement 104a comprises a flat spring formed of a strip or ribbon of spring material, and having windings that extend in a longitudinal direction as shown. The spring has a nested configuration shown in FIG. 9A. A stop within the lumen of the telescoping reinforcement 104a is coupled to the portion of the spring that forms the distal end of the telescoping reinforcement 104a when it is expanded. When the end effector of an instrument inserted into the instrument channel enters the telescoping reinforcement 104a, it engages the stop such that continued advancement of the end effector expands the reinforcement 104a to the FIG. 9B position. When force against the stop is released as a result of retraction of the instrument end effector, the spring retracts to the FIG. 9A position. It should be noted that the internalmost surface of the flat wire spring may engage with an insulating innermost member to prevent the spring from becoming hot while firing electro-cautery equipment inside the lumen. Additionally, the engagement feature on the flexible instrument may engage with the most proximal surface of the innermost wind on the flat wire spring. The innermost member against which the engagement feature exerts force to extend the spring may or may not be terminally bonded with the flat wire spring. The innermost member may allow be allowed to rotate inside the flat wire spring, to prevent undesirable torsion from being applied to the flat wire spring. Additionally, the engagement feature on the instrument end effector or shaft may be comprised of a bearing and shelf or other feature that allows smooth rotation of the instrument end effector or shaft without applying torsion to the innermost telescoping member.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed. Moreover, features of the various disclosed embodiment may be combined in a variety of ways to produce additional embodiments.

Any and all patents, patent applications and printed publications referred to above, including for purposes of priority, are incorporated herein by reference.

We claim:

1. An instrument channel device for receiving a medical instrument having a distal tip, the instrument channel device comprising:
an elongate tubular shaft including a proximal shaft portion and a distal shaft portion positionable within a body cavity, the shaft defining a lumen extending longitudinally therethrough for passage of the distal tip of the medical instrument therethrough and for positioning of the medical instrument such that its distal tip is disposed distally of the distal shaft portion;
a telescoping reinforcement positioned on the distal shaft portion, the reinforcement having a collapsed position and an expanded position, wherein in the collapsed position the telescoping reinforcement has a length that is shorter than the length of the telescoping reinforcement in the expanded position, the telescoping reinforcement engageable by a distal portion of the medical instrument during advancement of the distal tip distally from the distal shaft portion such that advancement of the distal tip to a position beyond a distal end of the telescoping reinforcement moves the telescoping reinforcement to the expanded position.

2. The instrument channel device of claim 1, wherein the telescoping reinforcement in the expanded position forms a generally rigid elongate member.

3. The instrument channel device of claim 1, wherein the telescoping reinforcement comprises a plurality of telescoping members, wherein in the first position the telescoping members are nested and wherein in the second position the telescoping members are longitudinally arranged.

4. The instrument channel of claim 2 wherein the members are rigid members.

5. The instrument channel of claim 1, wherein the telescoping reinforcement is retractable to the collapsed position by movement of the distal tip of the medical instrument proximally into the distal tip.

6. The instrument channel of claim 1, wherein the telescoping reinforcement comprises a telescoping coiled ribbon.

7. The instrument channel of claim 1, further including at least one retention element engaged with the telescoping reinforcement to retain the telescoping reinforcement in the retracted position.

8. The instrument channel of claim 3, further including a spring member having a first end coupled to a distal portion of the telescoping reinforcement, wherein the member is stretched when the telescoping reinforcement is in the expanded position and wherein, when the distal tip is withdrawn from the telescoping reinforcement, the spring retracts and draws the telescoping reinforcement proximally into the collapsed position.

9. A method for minimizing flexion of a distal end of a flexible instrument extending distally from an instrument channel, the method comprising:
providing an instrument channel device having a proximal shaft portion, a distal shaft portion, and a telescoping reinforcement on the distal shaft portion;
disposing the distal shaft portion within a body cavity;
inserting an instrument having a flexible shaft into a lumen of the instrument channel device;
advancing the distal tip of the instrument from a distal end of the lumen, such that advancement of the distal tip to a position beyond a distal end of the telescoping reinforcement moves the telescoping reinforcement to the expanded position, said reinforcement in said expanded position supporting the distal shaft portion disposed distal to the distal end of the lumen.
